(19) United States
(12) Patent Application Publication
(10) Pub. No.: US 2011/0117070 A1
(43) Pub. Date: May 19, 2011

(54) COMPOSITIONS AND METHODS FOR TREATING HEADACHE

(76) Inventors: Sheena K. Aurora, Seattle, WA (US); Curt Hendrix, Westlake Village, CA (US)

(21) Appl. No.: 12/844,700

(22) Filed: Jul. 27, 2010

Related U.S. Application Data
(63) Continuation-in-part of application No. 11/406,953, filed on Apr. 18, 2006.
(60) Provisional application No. 60/673,099, filed on Apr. 19, 2005.
(60) Provisional application No. 60/674,107, filed on Apr. 21, 2005.

(51) Int. Cl. A61K 33/08; A61K 31/525; A61K 31/357; A61K 31/365; A61K 36/28; A61P 25/06; A61P 25/00

(52) U.S. Cl. 424/94.1; 424/688; 514/251; 514/454; 424/725

(57) ABSTRACT

Compositions and methods for the treatment of headaches, such as migraine and cluster headaches, comprising co-administration of a first therapeutic component comprising topiramate and a second therapeutic component comprising a therapeutically effective amount or mixture of one or more therapeutic agents selected from the group consisting of riboflavin, magnesium, coenzyme Q10, petasin, isopetasin, and parthenolide. Also disclosed are methods for increasing the efficacy, and reducing or preventing side effects, of topiramate treatment.

This application is a continuation-in-part of U.S. patent application Ser. No. 11/406,953, filed Apr. 18, 2006, which claimed the benefit of U.S. Patent Application No. 60/673,099, filed Apr. 19, 2005 and U.S. Provisional Application No. 60/674,107, filed Apr. 21, 2005. The full disclosure of each of these patent applications is hereby incorporated by reference herein.

### BACKGROUND

The drug topiramate, a sulfamate-substituted monosaccharide, designated chemically as 2,3:4,5-Di-O-isopropylidene-β-D-fructopyranose sulfamate or 2,3:4,5-bis-O-(l-methylethylidene-β-D-fructopyranose sulfamate, having the structural formula:

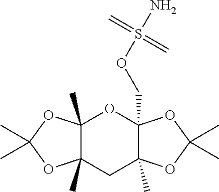

has been used and approved for use in the United States as an anticonvulsant for the treatment of epilepsy and Lennox-Gastaut syndrome in children. Dosages are usually 400 mg/day, with the drug administered orally in 2 doses. A schedule of gradually increasing dosages is generally recommended, typically starting from two 50 mg doses per day to two 200 mg doses per day over a 6 week period.

Topiramate has also been approved by the United States Food and Drug administration for the treatment of migraine headaches. Its exact mechanism of action is not certain, although it is known to affect neuronal excitability. For migraine, the recommended dosage begins at 25 mg per day increasing to the final recommended dosage of 50 mg twice per day.

Topiramate reduced migraine frequency among patients having an average of 5.5 migraine per month at a rate of about 2 headaches per month compared to a reduction of one headache per month with placebo. Multicentered, randomized, double-blind, placebo controlled, parallel group clinical trials of approximately 900 migraine sufferers, with open label studies on an additional 400 individuals, indicate that at 100 mg per day or greater, topiramate had statistically significant prophylactic effects versus placebo. By contrast, however, there was no statistical significance (i.e., no substantial reduction) reported in the reduction of migraine incidents at 50 mg/day or less of topiramate vs. placebo.

These studies also showed that patients receiving 100 mg or more per day of topiramate experienced significant unacceptable side effects including cognitive-related dysfunction (e.g. confusion, psychomotor slowing, difficulty with concentration/attention, memory loss, stupor, slurring of speech and word finding difficulty), depression and mood problems, somnolence, fatigue, parathesias, hyperventilation, anorexia, allergic reactions, chest pain, cardiac arrhythmias, liver malfunction, acute myopia, elevated ocular pressure, oligohydrosis and hyperthermia, among other symptoms.

Additionally, more then 1% of the patients reported an increase in migraine and other headaches as well as other unacceptable side effects and in broader commercial use additional side effects were noted. While these side effects can be minimized or reduced to acceptable levels by lowering the daily dosage the studies also indicated that at 50 mg per day, toprimate did not show statistically significant efficacy for migraine prophylaxis versus placebo at these dosages.

As a result of the fact that the efficacy of tobrimate and the occurrence of these side effects seem to be linked, many patients when provided with the higher 100 mg dosage and the prospect of these serious side effects either refuse to take topiramate or discontinue its use because of these side effects.

Ehrenberg, et al, U.S. Pat. Nos. 5,998,380 and 6,503,884 disclose the use of sulfamates, of which topiramate is one example, for treating migraines. However, the specific examples therein report on dosages of 600-800 mg/day in a first instance and 200-400 mg/day in a second instance of topiramate in order to obtain substantial reduction of migraine symptoms.

U.S. Pat. No. 6,319,903 describes the use of topiramate to treat cluster headaches. While the suggested dosage ranges from 15 mg to 1000 mg per day, preferably greater then 25 mg/day, the examples show that all dosages (50, 100, or 125 mg/day) were inadequate in providing rapid relief, and even with daily delivery in the 50-125 mg range it took 1-3 weeks to control the cluster headaches.

U.S. Pat. Nos. 6,559,293 and 6,699,840 disclose the use of salts of topiramate (preferably topiramate sodium) for the treatment of numerous physical conditions including neuropathic pain, migraine and cluster headaches.

U.S. Pat. Nos. 6,500,450 and 6,068,999 disclose and claim the use of compositions containing a) parthenolide, particularly parthenolide extracted from feverefew or feverfew including naturally-occurring parthenolide, b) riboflavin and c) magnesium provided by numerous different magnesium-containing chemical compositions, such as, magnesium in the form of acid salts, magnesium oxide, complexes or chelates. While these compositions have shown significant efficacy in treating and preventing migraines, they are not necessarily effective for all types of migraines or for other types of headaches.

Each and every publication cited in this patent application, including the patents and patent applications cited above, is hereby expressly and individually incorporated by reference herein in its entirety. However, none is hereby admitted to be prior art.

### SUMMARY OF THE INVENTION

The Applicant has discovered methods and compositions for treating a mammal, particularly a human, with amounts of topiramate and at least one other ingredient at dosages that are effective to treat headaches, and particularly migraine headaches, cluster headaches and the like. In a preferred embodiment, the combination of topiramate and a second therapeutic component may be administered in separate dosage forms. In other preferred embodiments, a first therapeutic component comprising topiramate and a second therapeutic component comprising at least one other agent may be administered in a single combined dosage form.

In a preferred composition the second therapeutic component includes at least one agent selected from the group consisting of parthenolide, magnesium and riboflavin. In another preferred embodiment, the second therapeutic component may comprise at least two agents selected from the group consisting of parthenolide, magnesium and riboflavin; in another preferred embodiment, the second therapeutic component may comprise at least three agents selected from the group consisting of parthenolide, magnesium and riboflavin.

In other embodiments described above and elsewhere in this patent application, the second therapeutic component comprises one or more agent selected from the group consisting of riboflavin, magnesium, coenzyme Q10, petasin, isopetasin, and parthenolide.

A particular advantage of the administration of topiramate with other effective ingredients is that this composition, and/or methods employing a first and second component as described above, is effective and reduces or eliminates the side effects experienced by users of prior topiramate compositions. For example, in a preferred embodiment of the present invention, an advantage of the present invention is that a minimum effective dose of at least one therapeutic agent is significantly lower when used in combination with at least one additional therapeutic agent. For example, the first therapeutic agent may comprise topiramate, and the second therapeutic agent may comprise at least one, or at least two, or at least three, or more therapeutic factors selected from the group consisting of riboflavin, magnesium, coenzyme Q10, petasin, isopetasin, and parthenolide, wherein a minimum migraine-relieving effective dose of topiramate is greater when this compound is used alone as compared to the minimum migraine-relieving effective dose of topiramate when this compound is used in combination with one or more of: riboflavin, magnesium, coenzyme Q10, petasin, isopetasin, and parthenolide.

In another embodiment, the first therapeutic agent may comprise at least one, or at least two, or at least three therapeutic factors selected from the group consisting of riboflavin, magnesium, coenzyme Q10, petasin, isopetasin, and parthenolide, and the second therapeutic agent may comprise topiramate, wherein a minimum migraine-relieving effective dose of one or more of: riboflavin, magnesium, coenzyme Q10, petasin, isopetasin, and parthenolide is greater when is used alone as compared to the minimum migraine-relieving effective dose of such agent or agents when used in combination with topiramate.

In other preferred embodiments the invention comprises methods of reducing side effects observed when treating a headache pain with a given dosage of topiramate comprising co-administering with said dosage of topiramate an effective amount of at least one of riboflavin, magnesium, coenzyme Q10, petasin, isopetasin, and parthenolide.

Presently a commercially available combination tablet sold under the name Migrelief® dietary supplement contains 50 mg puracol feverfew extract (comprising partheolide), 180 mg magnesium citrate/oxide (1:1), and 200 mg riboflavin. The maker of this dietary supplement recommends taking one tablet twice daily for the relief of migraine headache. The maker also discloses that there is a 90 day “build-up period” before maximum efficacy is achieved.

A second medicine used for headache relief has the trade name Migravent® natural supplement containing butterbur extract (at least 7.5 mg of petasin and isopetasin, which together help reduce spasms in cerebral blood vessels and inhibit the production of leukotrienes, compounds that trigger inflammation of blood vessels. Butterbur extract is known to have a spasmolytic effect. This means that it reduces spontaneous activity and spasms in the smooth muscular system, including the vascular walls. Migravent® also contains a blend of riboflavin, magnesium and coenzyme Q10; these combined ingredients are present in a “proprietary blend” of 876 mg.

The present invention involves a method for increasing the therapeutic efficacy of a given therapeutically effective dose of topiramate by administering said dose with one or more therapeutic agent selected from the group consisting of riboflavin, magnesium, coenzyme Q10, petasin, isopetasin, paracetamol, codeine, and parthenolide.

In a particularly preferred aspect of the presently disclosed methods and compositions the daily dosage of topiramate is from about 10 mg, or about 15 mg, or about 20 mg, or about 30 mg, to about 35 mg, or about 40 mg, or about 45 mg or about 50 mg, or to about 65 mg, or to about 100 mg or to about 125 mg, or about 200 mg or about 400 mg when used in combination with a second therapeutic component comprising a therapeutically effective amount or mixture of riboflavin, magnesium, coenzyme Q10, petasin, isopetasin, and parthenolide. Preferably, the second therapeutic component comprises a therapeutically effective amount, or mixture, of one or more of parthenolide, magnesium and riboflavin.

Preferably the magnesium, if present, is present as a salt and/or an oxide. Preferably if the second therapeutic component contains a magnesium salt, one or more of the following salts is present: magnesium citrate, magnesium acetate, magnesium formate, magnesium lactate or magnesium oxalate. In a preferred embodiment approximately a 1:1 ratio of magnesium oxide and the organic magnesium salt is used.

Parthenolide is usually extracted from natural sources. A particularly useful natural source of parthenolide is Feverfew (Chrysanthemum parthenium), from the Latin name of which the compound takes its name. It is a potential anticancer drug, and is known to destroy acute myelogenous leukemia cells by inducing apoptosis, leaving normal bone marrow cells relatively unscathed. Moreover, the compound may get at the root of the disease because it also kills stem cells that give rise to AML. Parthenolide is under investigation as a potential cancer drug in combination with sulindac. Parthenolide is known to be active against the parasite Leishmania amazonensis. It has anti-inflammatory and anti-hyperalgesic effects and blocks lipopolysaccharide-induced osteolysis. Parthenolide has limited solubility in water, and water soluble analogs that are easier to absorb are currently being sought. It has the following chemical structure:

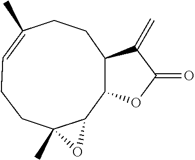

Petasin and isopetasin are found in the butterbur plants (Asterseae family, genus Petasites). These plants were used by Native Americans as a remedy for headache and inflammation, and have been the subject of various clinical studies as an effective preventative treatment for migraine. See e.g., Lipton, R. B., et al., NEUROLOGY, 63 (12):2240-2244 (Dec. 28, 2004). Petasin has the chemical structure:

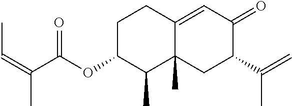

Isopetacin has the following chemical structure:

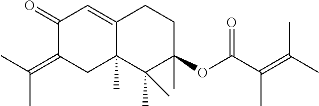

Thus, in a preferred embodiment of the present invention, a single composition (or the co-administration) of the first and second therapeutic components described above provides more effective prevention and treatment of more types of headaches than either a first therapeutic component containing a therapeutically effective amount of topiramate or the second component (containing a therapeutically effective amount or mixture of at least one of riboflavin, magnesium, coenzyme Q10, petasin, isopetasin, and parthenolide) when therapy includes just one of these components, and allows the previously used effective dosage of topiramate to be reduced, while maintaining the analgesic efficacy of a higher dosage of topiramate when used alone. However, in a preferred embodiment the present compositions and/or methods can be used with no, or significantly reduced, deleterious side effects commonly seen when using higher daily dosages of topiramate.

### DETAILED DESCRIPTION OF THE INVENTION

The present invention is directed to compositions and methods for the effective treatment of headaches, particularly, although not exclusively, migraine, cluster headaches, and mixtures of these types of headaches with sinus headache, tension headache, and or hormone headache. In particular, the present invention involves compositions and methods of administering two or more therapeutic components to a patient in need of headache treatment; in this method the efficacy of the treatment is greater than when one such component is used. In another preferred embodiment of the claimed method and/or composition, when a first therapeutic component comprises a topiramate component, the administration of a combination drug, or the co-administration of the two components separately, results in a decrease in side effects seen as compared to when the same dose of the topiramate component is administered alone.

Thus, in a first broad embodiment the invention concerns a composition for the treatment of headache comprising therapeutically effective amounts of first and second therapeutic components. In a very preferred embodiment the first therapeutic component comprises topiramate and the second therapeutic component comprises a therapeutic effective amount or mixture of one or more therapeutic agents selected form the group consisting of riboflavin, magnesium, coenzyme Q10, petasin, isopetasin, and parthenolide.

In a second broad embodiment the invention concerns a method for the treatment or prevention of headache comprising the administration to a patient of therapeutic effective amounts of first and second therapeutic components. In a very preferred embodiment the first therapeutic component comprises topiramate and the second therapeutic component comprises a therapeutically effective amount or mixture of one or more therapeutic agents selected from the group consisting of riboflavin, magnesium, coenzyme Q10, petasin, isopetasin, and parthenolide.

In another broad embodiment, the invention involves a method for decreasing the severity and/or number of side effects observed following administration of a first therapeutic component comprising a therapeutically effective amount of topiramate to a patient suffering from or at risk of suffering from headache without decreasing, or without substantially decreasing, the headache-relieving analgesic efficacy of the treatment. The method involves the administration of a first therapeutic component comprising a therapeutically effective amount of topiramate and a second therapeutically effective component comprising a therapeutically effective amount or mixture comprising one or more agent selected from the group consisting of riboflavin, magnesium, coenzyme Q10, petasin, isopetasin, and parthenolide. In this method the severity and/or number of side effects observed when the topiramate is administered with the second component is statistically less than when the same concentration of topiramate is used alone.

In a particularly preferred feature of the methods and compositions of the present invention, the second therapeutic component comprises a therapeutically effective mixture of at least two of: riboflavin, magnesium and parthenolide; in certain embodiments, the second therapeutic component comprises a therapeutically effective mixture of riboflavin, magnesium and parthenolide. The parthenolide may be present at a concentration of less than 0.7%, about 0.7%, or greater than 0.8%. The parthenolide may be present as part of an herbal extract, such as an extract from the feverfew plant.

As indicated above, the magnesium may contain a magnesium oxide. Preferably the magnesium includes a magnesium oxide and an organic magnesium salt. The ratio of magnesium oxide and the organic magnesium salt is preferably present at a ration of about 1:1. Alternatively the magnesium present in the oxide and the organic salt is present at a ratio of about 1:1.

In another preferred embodiment, the second therapeutic component of the present invention comprises one or more therapeutic agents selected from the group consisting of petasin, isopetasin, riboflavin, magnesium and coenzyme Q10. Preferably in this preferred embodiment the second therapeutic component comprises two or more or three or more therapeutic agent selected from the group consisting of petasin, isopetasin, riboflavin, magnesium and coenzyme Q10. In these aspects, preferably both petasin and isopetasin are present in a therapeutically effective blend or mixture. For example, these compounds may be present in an extract of the Butterbur plant. Preferably one or both of pertasin or isopetasin are present in this embodiment of the invention; the addition of magnesium would normally (although not exclusively) be the next preferred ingredient. Preferably all of these agents are present in this embodiment of the invention in a therapeutically effective mixture.

In this or any embodiment of the invention, either the first or second therapeutic component may comprise an antiemetic to treat the nausea and other gastrointestinal symptoms often associated with migraine. Such antiemetics may include, for example, prochloroeprazine, aldoperidol, promethazine, metoclopramine, chlorpromazine, trimethybenzamide, buclizine and ondansentron. Each of these agents has its own characteristics, and certain agents may be better for one patient than for another. For this reason, it may be preferred to administer the antiemetic as a separate medication dependent upon the patient's medical history and profile, if it is to be administered at all.

Various dosage ranges of topiramate may be used in the first therapeutic component of the present invention to help provide a therapeutically effective degree of headache analgesia. Thus, in a first concentration range, from about 10 mg to about 400 mg or more, or to about 300 mg, or to about 200 mg or to about 150 mg or to about 100 mg, or to about 75 mg, or to about 50 mg of topiramate may be effective as a daily dose when used in conjunction with a second therapeutic component containing a therapeutically effective amount or mixture of one or more agent selected from the group consisting of: riboflavin, magnesium, coenzyme Q10, petasin, isopetasin and parthenolide.

In a second concentration range, from about 25 mg to about 400 mg or more, or to about 300 mg, or to about 200 mg or to about 150 mg or to about 100 mg, or to about 75 mg, or to about 50 mg of topiramate may be effective as a daily dose when used in conjunction with a second therapeutic component containing a therapeutically effective amount or mixture of one or more agent selected from the group consisting of: riboflavin, magnesium, coenzyme Q10, petasin, isopetasin, and parthenolide.

In a third concentration range, from about 50 mg to about 400 mg or more, or to about 300 mg, or to about 200 mg or to about 150 mg or to about 100 mg, or to about 75 mg of topiramate may be effective as a daily dose when used in conjunction with a second therapeutic component containing a therapeutically effective amount or mixture of one or more agent selected from the group consisting of: riboflavin, magnesium, coenzyme Q10, petasin, isopetasin, and parthenolide.

In a fourth concentration range, from about 50 mg to about 400 mg or more, or to about 300 mg, or to about 200 mg or to about 150 mg or to about 100 mg, or to about 75 mg of topiramate may be effective as a daily dose when used in conjunction with a second therapeutic component containing a therapeutically effective amount or mixture of one or more agent selected from the group consisting of: riboflavin, magnesium, coenzyme Q10, petasin, isopetasin, and parthenolide.

In a fifth concentration range, from about 75 mg to about 400 mg or more, or to about 300 mg, or to about 200 mg or to about 150 mg or to about 100 mg of topiramate may be effective as a daily dose when used in conjunction with a second therapeutic component containing a therapeutically effective amount or mixture of one or more agent selected from the group consisting of: riboflavin, magnesium, coenzyme Q10, petasin, isopetasin, and parthenolide.

When the second therapeutic component comprises magnesium, magnesium may be provided by numerous different magnesium-containing chemical compositions, such as, magnesium in the form of acid salts, magnesium oxide, complexes or chelates; these compounds, complexes or chelates can provide patients with the same or better degree of relief from or prophylaxis of migraine (as well as other forms of headache) while significantly reducing the known side effects compared to treatment using the same dosage of topiramate as the sole analgesic agent.

Preferably, the second therapeutic component comprises magnesium oxide. If magnesium oxide is present, it is preferred that a second magnesium-containing compound, complex or chelate also be present at a range of ratios of from about 2:1 magnesium oxide to the other magnesium form to about 1:2 magnesium oxide to the other magnesium form. Preferably, the other form is a magnesium organic salt, such as magnesium citrate, magnesium acetate, magnesium formate, magnesium lactate or magnesium oxalate. In a preferred embodiment approximately a 1:1 ratio of magnesium oxide and the other form (preferably an organic magnesium salt) is used.

As used herein the term “parthenolide” includes, but is not limited to, parthenolide extracted from feverfew or other natural sources of parthenolide, or the use of feverfew or other natural products including naturally occurring parthenolide, as well as synthetic parthenolides, chemically modified parthenolide or parthenolide derivates such as esters and other prodrugs. As used herein the term “prodrug” means a compound which is administered or ingested in an inactive form or in a form of reduced activity, and which metabolizes to a fully active form of the drug in vivo by, for example, the hydrolysis of an ester linkage or another labile bond by endogenous enzymes.

### Combination Drugs

In certain embodiments of the present invention, the first therapeutic component and the second therapeutic component are provided in a single composition as a combination dosage form.

The dosage form can be any suitable and effective dosage form, including, without limitation, a tablet, a capsule, a powder, a liquid, a transdermal patch, a nasal dosage form, a biocompatible (such as a biodegradable) implant, formulation for injection, and suppository. Preferably the formulation may further be formulated for extended or delayed release, such as through coatings and coordination with a chelate, formulation within spheres or granules containing permeable or biodegradable coatings, coatings having lower specific gravity than gastric juice, and similar coatings and methods of extending and delaying release of all, or a portion, of an administered active ingredient or ingredients. Such coatings may include cellulose derivatives, such as carboxymethyl cellulose, hydroxypropylmethyl celloluse, hydroxypropylcellulose, ethylcellulose, methylcellulose, microcrystalline cellulose and carrageenan and the like; methyacrylic acid/methacrylate esters, such as anionic and cationic polymers of methacrylic acid, copolymers of methacrylate, copolymers of acrylates and methacrylates, and copolymers of ethacrylate and methylmethacrylate; polyvinylacetate phthalate; and shellac.

In certain embodiments oral or suppository dosage forms may comprise a combination of semi permeable and osmotic tablet core to produce a “zero order” release technology to provide an extended release dosage form.

Certain forms of the compositions of the present invention, for example, may comprise a coating, shell, or core, or a monolithic form of a biodegradable polymer. The biodegradable polymer may, for example, comprise a biodegradable polylactate component, a polyglycolate component, or a poly(lactate)-poly-(glycolate) copolymer component.

Whatever the type of dosage form is required and used, in one embodiment the invention comprises a single doage form comprising a single therapeutic component containing a therapeutically effective amount of topiramate and a therapeutically effective amount of a second therapeutic component comprising a therapeutic amount of a mixture of one or more therapeutic agents selected from the group consisting of riboflavin, magnesium, coenzyme Q10, petasin, isopetasin, and parthenolide.

### Methods of Treatment

In another embodiment of the present invention the invention is drawn to methods of treating headache (such as, without limitation, migraine headache, cluster headache, tension headache, and mixtures of these types of headaches with sinus headache, tension headache, and/or hormone headache. For example, the present invention involves methods of administering two or more analgesic therapeutic components to a patient in need of headache treatment; in this method the efficacy of the treatment is greater than when an effective dose of only one of these therapeutic components is used. Preferably, when a first therapeutic component comprises topiramate, the topiramate-related side effects are unexpectedly reduced when a second therapeutic component is co-administered, relative to administration of the same amount of the topiramate component alone. By “co-administered” is meant that the patient is simultaneously adhering to a therapeutic regimen involving the administration of a first therapeutic component and a second therapeutic component. Thus, this may involve the administration of a single dosage form containing first and second therapeutic components, or such methods may involve the co-administration of a first therapeutic component and a second therapeutic component as separate dosage forms. If separate dosage forms, the first and second therapeutic components may be co-administered at substantially the same time or at different times.

Thus in another preferred embodiment the present patent application is directed to methods of reducing at least one side effect of a topiramate component administered to a patient suffering from, or at risk of suffering from, a headache when said topiramate component is co-administered in a headache-reducing therapeutic amount in a first therapeutic component together with a second therapeutic component comprising a therapeutically effective amount of mixture of one or more therapeutic agent selected form the group consisting of riboflavin, magnesium, coenzyme Q10, petasin, isopetasin, and parthenolide. The one or more side effects may include, without limitation, cognitive-related dysfunction (e.g. confusion, psychomotor slowing, difficulty with concentration/attention, memory loss, stupor, slurring of speech and word finding difficulty), depression and mood problems, somnolence, fatigue, parathesias, hyperventilation, anorexia, allergic reactions, chest pain, cardiac arrhythmias, liver malfunction, acute myopia, elevated ocular pressure, oligohydrosis and hyperthermia. In one embodiment, the number of side effects are reduced upon co-administration of the first therapeutic component with a second therapeutic component relative to administration of the same amount of the active agent or agents in the first therapeutic component alone (without the second component) under otherwise identical conditions, such as dosage form, time of day, volume, route of administration, severity of headache, and so forth. Preferably the first therapeutic component comprises a topiramate component. And as indicated above, in this method the first and second therapeutic components may comprise a single dosage form or separate dosage forms. If separate dosage forms, the first and second therapeutic components may be co-administered at substantially the same time or at different times.

The composition and methods of the present invention also comprise improved methods for treating a headache (such as a migraine, cluster or other severe headache), or as a prophylactic to prevent the occurrence of a headache. Preferred compositions comprise a combination including therapeutically effective amount of a first therapeutic component comprising a topiramate component and a second therapeutic component comprising the combination of agents set forth in U.S. Pat. Nos. 6,500,450 and 6,068,999, hereby incorporated by reference herein. When an effective therapeutic dose in a concentration range, for example, from about 10 mg to about 400 mg or more, or from about 10 mg to about 300 mg, or from about 10 mg to about 200 mg or from about 10 mg to about 150 mg or from about 10 mg to about 100 mg, or from about 10 mg to about 75 mg, or from about 10 mg to about 50 mg of topiramate is delivered in combination with therapeutic amounts or mixtures of one or more of parthenolide, magnesium and riboflavin as set forth herein, it may be possible to also reduce the dosage of the parthenolide, magnesium and/or riboflavin (for example, essentially as set forth in the '999 and '450 patent) and still achieve equal or superior treatment of migraine, cluster, or other headaches, and reduction of the systemic symptoms commonly experienced by migraine suffers (nausea, sensitivity to light, aura, blurred or distorted vision, feelings of numbness in the body, throbbing sensations, etc.) relative to administration of the same amount of the topiramate in the first therapeutic component alone (without the second component) under otherwise identical conditions, such as dosage form, time of day, volume, route of administration, severity of headache, and so forth.

The compositions and methods set forth herein will allow both physicians who treat chronic migraine patients and headache suffers, and the patients themselves, to effectively reduce the frequency of chronic migraine symptoms and/or reduce side effects, thus allowing continued use of this therapy because of enhanced tolerability by the patient for the active ingredient(s), such as a topiramate component.

Preferred compositions for daily delivery, which may be provided in single or multiple dosages over a 24 hour period, may comprise an effective dose of topiramate in combination with one or more of the following: 10-1000 mg of magnesium from any source, 10-1000 mg of riboflavin, and 0.1 to about 30 mg of parthenolide, for example as contained as part of the herb feverfew. The feverfew (generally 10-1000 mg thereof) may be administered in the form of powdered whole herb, tinctures, extracts or other forms containing from about of parthenolide.

In one embodiment the preferred total dosage in a 24 hour period is 10 mg-75 mg topiramate, and one or more of: 200 mg-400 mg riboflavin, 300 mg-600 mg of magnesium and at least 0.2 mg of parthenolide, preferably delivered in two or three equal dosages spaced over that 24 hour period. The magnesium is preferably delivered as acid salts, complexes or chelates of magnesium and may also include magnesium oxide.

Added benefits can be obtained by also adding one or more of: CoQ-10 (for example, about 10 mg to about 500 mg); 200 micrograms to about 20 mg of any source of folates or folate metabolites or derivatives (for example, about 200 micrograms to about 20 mg); vitamin B-6 or its derivatives or metabolites (for example, about 10 mg to about 500 mg); vitamin B-12 (about 100 micrograms to about 5 mg), the vitamin B-12 including but not limited to cyanobobalmin, hydroxcobalamin, methycobalamin, and adenosylcobalamin; from about 10 mg to about 10 grams of carnitine from various sources including 1-carnitine, and acetyl carnitine; about 250 mg to about 10 grams of taurine; NMDA antagonists, such as about 10 mg to about 3 grams acetylcysteine or abut 5 mg to 1 grams DL-2-aminophophonovaleric acid (APV); about 100 mg to about 3 grams carnosine; from about 50 mg to about 4 grams omega 3 essential fatty acids; from about 0.25 mg to about 15 mg of melatonin; from about 10 mg to about 1 gram of 5-HTP; from about 5 mg to about 1 gram 5-HT; from about 0.5 grams to about 10 grams EPA or DHA; from about 500 mg to about 10 grams GABA; from about 100 mg to about 3 grams ginger; from about 100 mg to about 2.5 grams niacin, particularly as niacinamide or nicotinic acid; from about 100 mg to about 3 grams N-acetyl cysteine; from about 50 micrograms to about 5 mg of selenium; from about 100 mg top about 3 grams potassium; and from about 100 mg to 5 grams of carnosine.

### EXAMPLES

### Example 1-20

In a headache clinic, 20 patients suffering from migraine are treated with a dosage of topiramate and then an additional, secondary therapeutic component comprising one of the following therapeutic agents, or mixture of agents: 1) a commercially available combination tablet sold under the name Migrelief® dietary supplement contains puracol feverfew extract (comprising partheolide), magnesium citrate/oxide (1:1), and riboflavin; and 2) a second medicine having the trade name Migravent® natural supplement containing butterbur extract (including petasin and isopetasin), and a blend of riboflavin, magnesium and coenzyme Q10; these combined ingredients are present in a “proprietary blend” of 876 mg.

In twenty patients suffering from migraine the effect of augmentation of topiramate treatment was formally documented. Table 1 shows the effectiveness and efficacy of a course of migraine treatment with the indicated daily dosage of topiramate alone; patients were administered topiramate alone in monotherapy for at least 6 weeks before side effect and efficacy data were obtained. Table 2 shows the change in efficacy and the side effect profile of the indicated dosage of topiramate when a second therapeutic component was also administered. Patients were treated in combination therapy with topiramate and the indicated second agent for 6 weeks before side effect and efficacy data were obtained for comparison with the monotherapy data. Dosages of the second therapeutic component was as follows: Migravent: 150 mg pyrrolizidine alkaloids (PA) free) and 876 mg (Riboflavin, Magnesium, Co-enzyme Q-10 [propriety blend]); Migralief: Magnesium citrate and oxide 360 mg, riboflavin 400 mg and Puracol Feverfew (Tanacetum parthenium).

The scale used to list efficacy is as follows: 1=no measurable efficacy; 2=some measurable difference in severity and or frequency of headache; 3=modest improvement in severity or frequency of headache; 4=headache frequency improvement either 50% or greater in frequency, severity or both;

The scale used to list the topiramate side effect profile was as follows: 1=mild or none; 2=tolerable, not treatment limiting; 3=warrants trial of topiramate dose reduction; 4=considering stopping topiramate treatment strongly.

The results of a retrospective chart review of the efficacy and side effect profile of topiramate treatment alone (Table 1) and administration with topiramate plus a second therapeutic component (Table 2) are shown below.

TABLE 1

| Patient Initials | Dosage of Topiramate | Efficacy | Side Effects |
|---|---|---|---|
| MA | 75 | 3 | 3 |
| VC | 100 | 4 | 4 |
| AD | 100 | 2 | 1 |
| LJ | 125 | 2 | 2 |
| SM | 50 | 2 | 3 |
| LS | 125 | 2 | 3 |
| LS | 400 | 2 | 1 |
| JS | 100 | 3 | 4 |
| TT | 75 | 3 | 3 |
| KW | 100 | 3 | 3 |
| AW | 50 | 2 | 3 |
| GV | 100 | 4 | 4 |
| WB | 150 | 3 | 1 |
| SC | 200 | 3 | 4 |
| LS | 100 | 2 | 4 |
| JF | 100 | 4 | 4 |
| IR | 75 | 3 | 4 |
| WR | 100 | 2 | 2 |
| ML | 50 | 2 | 1 |
| KH | 100 | 3 | 4 |

TABLE 2

| Patient | Additional | | | Dose Change |
|---|---|---|---|---|
| Initials | Agent | Efficacy | Side-Effect | (mg topiramate) |
| MA | Migralief | 4 | 1 | none |
| VC | Migralief | 4 | 1 | 75 mg |
| AD | Migralief | 4 | 1 | none |
| LJ | Migralief | 4 | 1 | none |
| SM | Migralief | 4 | 1 | 75 |
| LS | Migralief | 4 | 1 | 100 |
| LS | Migralief | 4 | 1 | none |
| JS | Migralief | 4 | 1 | none |
| TT | Migralief | 4 | 2 | none |
| KW | Migralief | 4 | 2 | none |
| AW | Migralief | 3 | 1 | none |
| GV | Migralief | 4 | 2 | 75 |
| WB | Migralief | 4 | 1 | 100 |
| SC | Migralief | 3 | 1 | 175 |
| LS | Migralief | 4 | 1 | 150 |
| JF | Migralief | 4 | 1 | 75 |
| IR | Migravent | 4 | 1 | none |
| WR | Migravent | 4 | 2 | none |
| ML | Migravent | 4 | 1 | 100 |
| KH | Migravent | 4 | 1 | 50 |

A comparison of Table 1 and Table 2 shows that in almost every case the efficacy of a given dose of topiramate improved, in terms of reducing the frequency and or severity of migraine. Additionally, in most of these twenty patients there were significantly fewer or less severe side effects experienced after initiation of a regimen of co-administration with the second therapeutic component, even when topiramate was used at the same dosage as it had previously been when administered alone. In some patients the dosage of topiramate could be lowered when co-administered with the second therapeutic component without jeopardizing the efficacy of treatment.

The invention is not limited by the foregoing disclosure or examples, which are only intended to illustrate the invention. Each ingredient, concentration, ratio, and temperature range given is expressly intended to include each and every ingredient, concentration, ratio, and temperature range included within it. The invention is defined solely by the claims that conclude this specification. Each and every publication mentioned or cited in this specification is hereby incorporated by reference herein in its entirety.

What is claimed is:

1) A composition for treating migraine, reducing the incidence of migraine, or reducing the severity of migraine comprising a daily dosage of a combination of from about 35 mg to about 50 mg of topiramate in combination with magnesium.

2) A therapeutic composition containing:
a first therapeutic component comprising an amount of topiramate in an analgesic-effective or prophylactic-effective amount, and
a second therapeutic component comprising a therapeutically effective amount or mixture of one or more therapeutic agents selected from the group consisting of riboflavin, magnesium, coenzyme Q10, petasin, isopetasin, and parthenolide.

3) The therapeutic composition of claim 2 wherein the topiramate is present in a concentration of from about 10 mg to about 400 mg.

4) The therapeutic composition of claim 2 wherein the topiramate is present in a concentration of from about 10 mg to about 150 mg.

5) The therapeutic composition of claim 2 wherein the topiramate is present in a concentration of from about 10 mg to about 100 mg.

6) The therapeutic composition of claim 2 wherein the second therapeutic component comprises a therapeutically effective amount or mixture of one or more of parthenolide, magnesium, and riboflavin.

7) The therapeutic composition of claim 2 wherein the first therapeutic component comprises from about 10 mg to about 150 mg of topiramate and the second therapeutic component comprises a therapeutically effective amount or mixture of parthenolide, magnesium, and riboflavin.

8) The therapeutic composition of claim 7 wherein the magnesium component comprises a combination of a magnesium oxide and an organic magnesium salt.

9) The therapeutic composition of claim 7 wherein the magnesium oxide and magnesium organic salt are present at a molar ratio of approximately 1:1.

10) The therapeutic composition of claim 2 wherein the second therapeutic component comprises an effective amount or mixture of at least one of a butterbur extract component, a Coenzyme Q10 component, riboflavin and magnesium.

11) The therapeutic composition of claim 10 wherein the second therapeutic component comprises a butterbur component containing an effective amount or mixture of at least one of petasin and isopetasin.

12) The therapeutic composition of claim 11 wherein the second therapeutic component comprises a mixture of at least about 7.5 mg petasin and isopetasin.

13) The therapeutic composition of claim 10 wherein the second therapeutic component comprises a coenzyme Q10 component.

14) The therapeutic component of claim 10 wherein the second therapeutic component comprises a therapeutically effective mixture of petasin and isopetasin, and a blend of riboflavin, magnesium and coenzyme Q10.

15) A method of treating or preventing a headache comprising the co-administration of a first therapeutic component comprising an amount of topiramate in an analgesic-effective or prophylactic-effective amount, and
a second therapeutic component comprising a therapeutically effective amount or mixture of one or more therapeutic agents selected from the group consisting of riboflavin, magnesium, coenzyme Q10, petasin, isopetasin, and parthenolide.

16) The method of claim 15 wherein the first therapeutic component comprises from about 10 mg to about 150 mg tobiramate.

17) The method of claim 16 wherein the second therapeutic component comprises a therapeutically effective amount or mixture of one or more of parthenolide, magnesium, and riboflavin.

18) The method of claim 17 wherein the second therapeutic component comprises a therapeutically effective amount or mixture of parthenolide, magnesium, and riboflavin.

19) The method of claim 18 wherein the second therapeutic composition comprises a combination of a magnesium oxide and an organic magnesium salt.

20) The method of claim 19 wherein the magnesium oxide and magnesium organic salt are present at a molar ratio of approximately 1:1.

21) The method of claim 15 wherein the antiemetic is a buclizine component.

22) The method of claim 15 wherein the second therapeutic component comprises an effective amount or mixture of at least one of a butterbur extract component, a Coenzyme Q10 component, riboflavin and magnesium.

23) The method of claim 22 wherein the second therapeutic component comprises a butterbur component containing an effective amount or mixture of at least one of petasin and isopetasin.

24) The method of claim 23 wherein the second therapeutic component comprises a mixture of at least about 7.5 mg petasin and isopetasin.

25) The method of claim 22 wherein the second therapeutic component comprises a coenzyme Q10 component.

26) The method of claim 22 wherein the second therapeutic component comprises a therapeutically effective mixture of petasin and isopetasin, and a blend of riboflavin, magnesium and coenzyme Q10.

27) A method for preventing, reducing or eliminating at least one side effect otherwise observed in a patient upon administration of a first dosage of a first therapeutic component comprising topiramate, comprising:
co-administering to said patient a second therapeutic component comprising a therapeutically effective amount or mixture of one or more therapeutic agents selected from the group consisting of riboflavin, magnesium, coenzyme Q10, petasin, isopetasin, and parthenolide,
wherein said at least one side effect is prevented, reduced or eliminated when said second therapeutic component is co-administered with the first dosage of topiramate compared to when said first dosage of topiramate is administered without said second component.
